(19) United States
(12) Patent Application Publication
(10) Pub. No.: US 2016/0022124 A1
(43) Pub. Date: Jan. 28, 2016

(54) CAPSULE ENDOSCOPE CONTROL DEVICE AND SYSTEM

(71) Applicant: SHENZHEN JIFU TECHNOLOGY CO.,LTD, ShenZhen (CN)

(72) Inventors: Yi LI, ShenZhen (CN); Ping SUN, ShenZhen (CN); Wenjun DENG, ShenZhen (CN); Jianping WANG, ShenZhen (CN)

(73) Assignee: SHENZHEN JIFU TECHNOLOGY CO.,LTD, ShenZhen (CN)

(21) Appl. No.: 14/799,731

(22) Filed: Jul. 15, 2015

(30) Foreign Application Priority Data
Jul. 23, 2014 (CN) 201410353836.X
Aug. 21, 2014 (CN) 201410416049.5

(51) Int. Cl. A61B 1/00; A61B 1/04

(52) U.S. Cl. CPC A61B 1/00158; A61B 1/041

(57) ABSTRACT

The present invention applies to the field of medical devices, and provides capsule endoscope control device and system. The capsule endoscope control device comprises: a frame; a rotating apparatus, disposed on the frame; a movement arm, secured to the rotating apparatus, a tail end of the movement arm being provided with a permanent magnet; and a driving apparatus, electrically connected to the rotating apparatus and the movement arm, and configured to receive an external movement control instruction, drive the rotating apparatus, the movement arm and the permanent magnet to move, and control position and posture of the capsule endoscope in a human body via a magnetic force of the permanent magnet. According to the present invention, by means of electrical control, the driving apparatus controls movement of the rotating apparatus and the movement arm, the permanent magnet disposed on the movement arm controls position and posture of the capsule endoscope in the human body, such that the capsule endoscope picks up complete images on the stomach, and accuracy and precision of medical diagnosis are improved.

### TECHNICAL FIELD

The present invention relates to the field of medical devices, and in particular, to capsule endoscope control device and system.

### BACKGROUND

A capsule endoscope has the advantage of monitoring and diagnosis without pain and gash, which is gradually applied to clinical diagnosis for various diseases. When a capsule endoscope is taken by an examinee, the capsule endoscope enters the stomach of the examinee. Relevant data is collected by using a lens component or a sensor to make clinical diagnosis, thereby reducing clinical pain of the examinee.

When entering the stomach of the examinee, the capsule endoscope can freely operate. The position of the capsule endoscope is uncertain, and the collected data is arbitrary. In this case, whether the capsule endoscope collects all data in a target region of the stomach cannot be determined. Accordingly, it is hard to judge the condition of the examination region of the stomach. Therefore, how to effectively control positions and postures of the capsule endoscope in the human body is very important for picking up desired images for the stomach.

### SUMMARY

Embodiments of the present invention provide a capsule endoscope control system, which is capable of effectively controlling position and posture of the capsule endoscope in the human body, and picking up complete images on the stomach.

An embodiment of the present invention provides a capsule endoscope control device, comprising:

a frame;

a rotating apparatus, disposed on the frame;

a movement arm, secured to the rotating apparatus, a tail end of the movement arm being provided with a permanent magnet; and

a driving apparatus, electrically connected to the rotating apparatus and the movement arm, and configured to receive an external movement control instruction, drive the rotating apparatus, the movement arm and the permanent magnet to move, and control position and posture of the capsule endoscope in a human body via a magnetic force of the permanent magnet.

An embodiment of the present invention further provides a capsule endoscope control system, comprising:

a frame;

a rotating apparatus, disposed on the frame;

a movement arm, secured to the rotating apparatus, a tail end of the movement arm being provided with a permanent magnet;

a driving apparatus, electrically connected to the rotating apparatus and the movement arm, and configured to receive an external movement control instruction, drive the rotating apparatus, the movement arm and the permanent magnet to move, and control position and posture of the capsule endoscope in a human body via a magnetic force of the permanent magnet; and

a control apparatus, communicated with the driving apparatus, and configured to provide an operation interface fir a user, transmit a movement control instruction of the user to the driving apparatus, store and process image data collected by the capsule endoscope from the human body.

According to the embodiments of the present invention, by means of electrical control, the driving apparatus controls movement of the rotating apparatus and the movement arm, the permanent magnet disposed on the movement arm controls position and posture of the capsule endoscope in the human body, such that the capsule endoscope picks up complete images on the stomach, and accuracy and precision of medical diagnosis are improved.

### BRIEF DESCRIPTION OF DRAWINGS

FIG. 1 is a schematic structural views of a capsule endoscope control system according to an embodiment of the present invention;

FIGS. 2 and 3 are schematic structural views of a capsule endoscope control device according to an embodiment of the present invention;

FIG. 4 is a schematic structural rear view of a capsule endoscope control device according to an embodiment of the present invention;

FIG. 5 is a schematic sectional view taken along an S-S direction in FIG. 4;

FIG. 6 is a schematic structural view of a capsule endoscope control device covered with a cylinder according to an embodiment of the present invention;

FIG. 7 is a schematic structural view of a control apparatus in a capsule endoscope control system according to an embodiment of the present invention;

FIG. 8 is a schematic structural view of a driving apparatus in a capsule endoscope control device according to an embodiment of the present invention;

FIG. 9 is a schematic internal structural view of a capsule endoscope according to an embodiment of the present invention;

FIG. 10 is a schematic view of the side of the capsule endoscope with a lens close to an inner wall of the human body according to an embodiment of the present invention;

FIG. 11 is a schematic view of the side of the capsule endoscope without a lens close to an inner wall of the human body according to an embodiment of the present invention; and

FIG. 12 is a schematic view of a capsule endoscope picking up images for an inner wall of the human body in different postures of a permanent magnet according to an embodiment of the present invention.

### DETAILED DESCRIPTION

To make the objective, technical solution, and advantages of the present invention more clear, the following section describes the technical solutions of the present invention in combination with the accompanying drawings and embodiments. It should be understood that the embodiments described here are only exemplary ones for illustrating the present invention, and are not intended to limit the present invention.

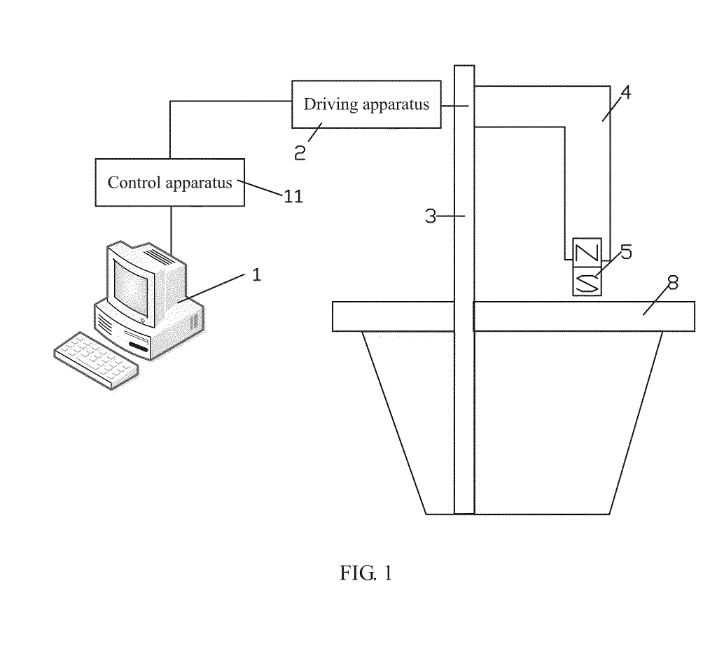

FIG. 1 illustrates a structure of a capsule endoscope control system according to an embodiment of the present invention. For ease of description, parts relevant to the embodiments of the present invention are only illustrated.

In the embodiment of the present invention, the capsule endoscope control system comprises a control apparatus 11 running on a terminal 1, and a capsule endoscope control device controllable by the control device 11.

The capsule endoscope control device comprises: a frame 30, a rotating apparatus 3, a movement arm 4, a permanent magnet 5, and a driving apparatus 2.

The rotating apparatus 3 is disposed on the frame 30.

The movement arm 4 is secured to the rotating apparatus 3, and a tail end of the movement arm 4 is provided with the permanent magnet 5.

The driving apparatus 2 is electrically connected to the rotating apparatus 3 and the movement arm 4, and configured to receive an external movement control instruction, drive the rotating apparatus 3, the movement arm 4 and the permanent magnet 5 to move, and control position and posture of the capsule endoscope in a human body via a magnetic force of the permanent magnet 5.

The control apparatus 11 running on the terminal 1 is communicated with the driving apparatus 2, and configured to provide an operation interface for a user, transmit a movement control instruction of the user to the driving apparatus 2, store and process image data collected by the capsule endoscope from the human body.

The control apparatus 11 is communicated with the capsule endoscope in a wireless manner, and may be communicated with the driving apparatus 2 in a wired manner or in a wireless manner.

In the embodiment of the present invention, the capsule endoscope control device employs an electrical control manner, and the driving apparatus 2 controls movement of the rotating apparatus 3 and the movement arm 4. Since the movement arm 4 is secured to the rotating apparatus 3, when the rotating apparatus 3 rotates, the movement arm 4 is driven to rotate as well, and meanwhile position and posture of the permanent magnet 5 are changed by regulating posture of the movement arm 4, thereby ensuring that the permanent magnet 5 is capable of traversing all positions in an examination region.

Referring to FIG. 1 to FIG. 3, the frame 30 comprises a base 301 supported on the ground, and a back plate 302 perpendicularly secured to the base 301 along a longitudinal direction. The rotating apparatus 3 is secured in an overlay manner to the back plate 302. The back plate 302 is provided with a through hole.

One end of the movement arm 4 is secured to a side surface of the rotating apparatus 3, and the other end of the movement arm 4 telescopically rotatably extends along a transverse direction.

The permanent magnet 5 is disposed at a tail end of the movement arm 4. When the capsule endoscope is taken by an examinee, the taken capsule endoscope is controlled by the permanent magnet 5.

The driving apparatus 2 is electrically connected to the rotating apparatus 3 and the movement arm 4, and configured to electrically control the rotating apparatus 3, the movement arm 4, and the permanent magnet 5.

There are multiple motors 32, which are separately disposed on the rotating apparatus 3 and the movement arm 4, and enables, according to the control instruction sent by the terminal 1 to the driving apparatus 2, the driving apparatus 2 to adjust the positions of the rotating apparatus 3 and the movement arm 4 so as to drive the permanent magnet 5 to reach the designated examination region.

The rotating apparatus 3 is in a hollow disk shape, and forms a receiving chamber 303 with a through hole opened on the back plate 302.

The receiving chamber 303 is configured to accommodate an examinee and is in a cylinder shape, is communicated with the rotating apparatus 3 and the back plate 302, and extends towards a stretching direction of the movement arm 4.

One end of the movement arm 4 is vertically secured to the rotating apparatus 3, and the other end of the movement arm 4 is movable on the outer side of the receiving chamber 303. There are multiple arm rods 41 that are sequentially connected.

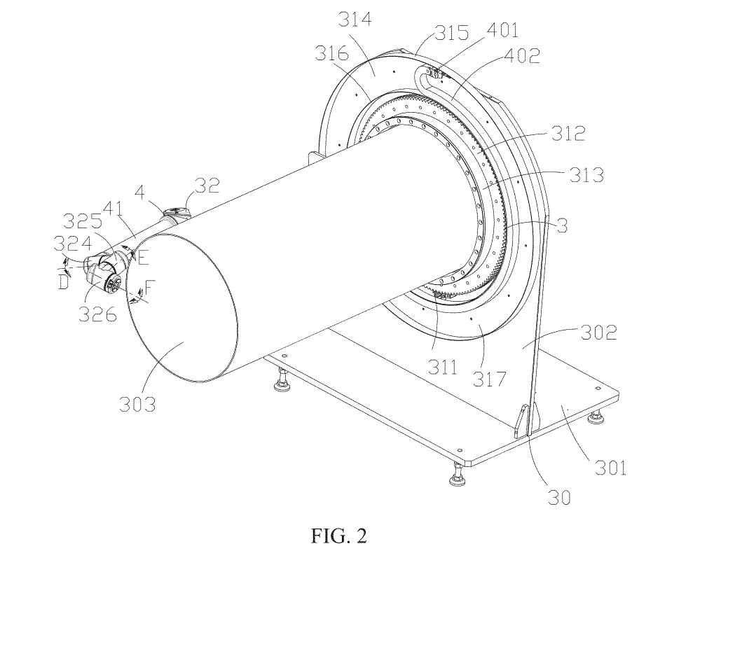

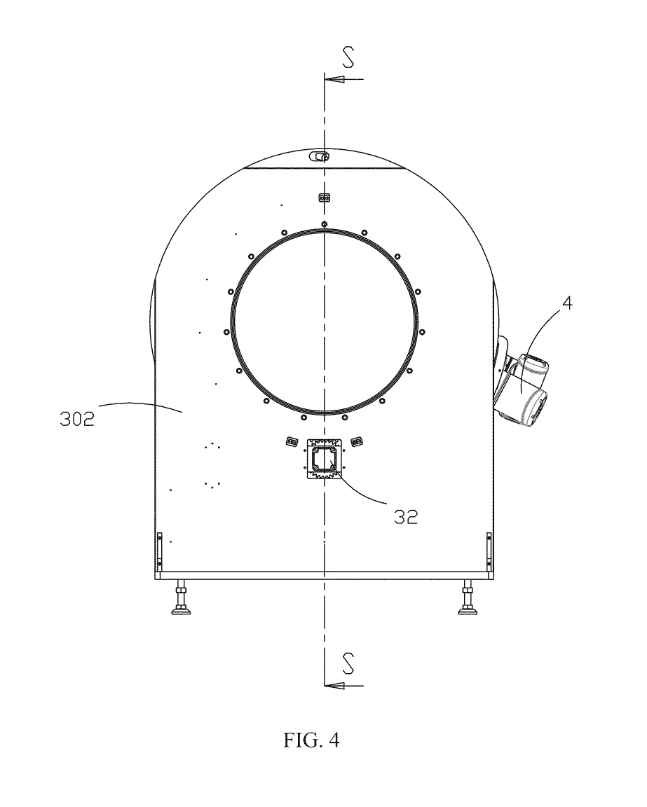

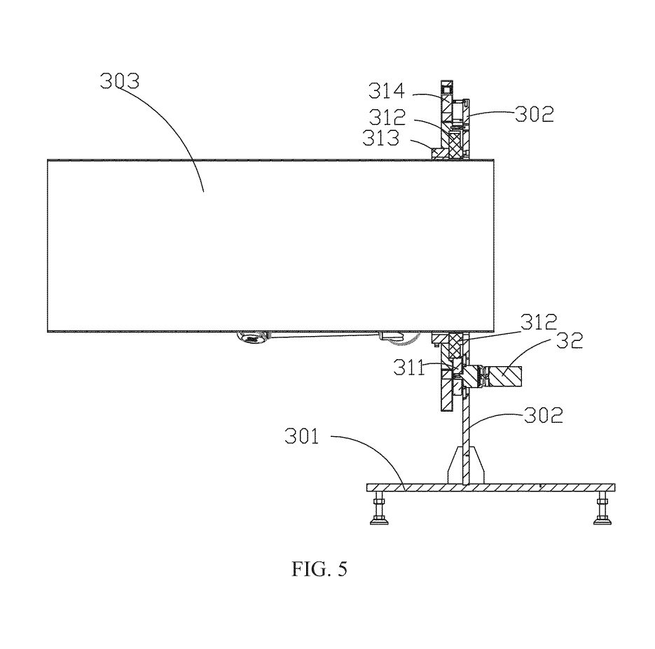

Referring to FIG. 2 to FIG. 5, the hollow disk-shaped rotating apparatus 3 comprises a driving wheel 311, a driven wheel 312, and a bearing 313.

The driving wheel 311 is securely disposed on one side at the bottom of the back plate 302, and a motor 32 connected to the driving wheel 311 is disposed on the other side of the back plate 302, wherein the motor 32 is configured to drive the driving wheel 311.

The driven wheel 312 is engaged with the driving wheel 311, and is rotatably connected to an outer ring of the bearing 313.

The bearing 313 is in hollow annular shape, and is secured to the back plate 302. The receiving chamber 303 is communicated with the bearing 313 and the back plate 302.

A guiding plate 314 is secured in an overlay manner to one side of the back plate 302. The guiding plate 314 is positioned on an outer ring at the bottom of the driven wheel 312.

An outer edge of the guiding plate 314 extends towards one side of the movement aim 4 to define a circular baffle 315, and a protrusion portion 316 being formed upwardly in a position close to the driven wheel 312 on an inner ring of the guiding plate 314. The baffle 315, the guiding plate 314, and the protrusion portion 316 define an annular guiding groove 317 therebetween.

When the rotating apparatus 3 drives the movement arm 4 to rotate, the activity space of a connection cable 401 of the movement arm 4 is provided. The connection cable 401 may be a signal cable and a power cable of the movement arm 4. To make the connection cable 401 tidy and beautiful, the connection cable 401 is sleeved. with a drag chain 402. The drag chain 402 may be freely bent, and movable in the guiding groove 317.

An installation plate 318 is secured to a top surface of the driven wheel 312, and one end of the movement arm 4 is secured to the installation plate 318. When the driven wheel 312 rotates, the installation plate 318 rotates accordingly and drives the movement arm 4 to rotate.

The installation plate 318 is in a planar structure, and spans over the guiding groove 317. One end of the installation plate 318 is secured to the driven wheel 312, and the other end extends outside the circular baffle 315, such that the installation plate 318 will not disturb the drag chain 402 in the guiding groove 317 during the process of rotating.

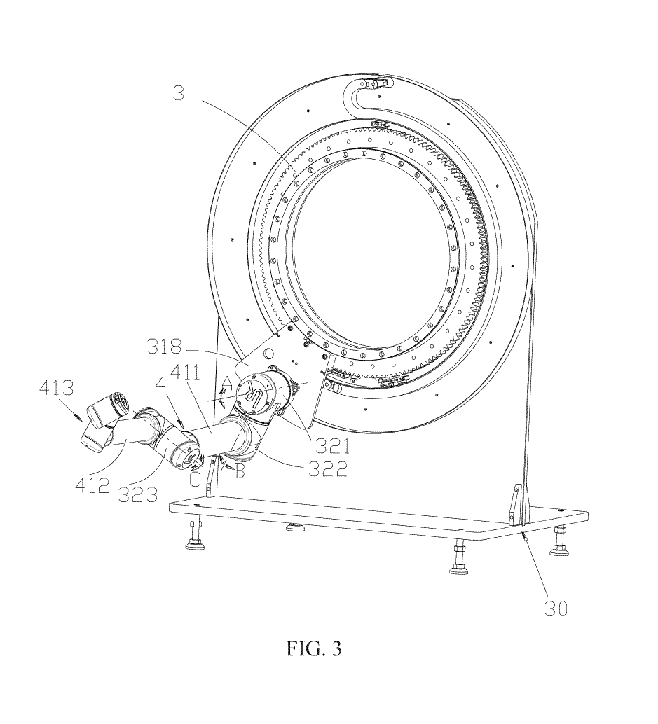

Referring to FIGS. 2 and 3, the movement arm 4 has at least two arm rods 41 connected with each other. The motor 32 is separately disposed between the arm rods 41, and between the arm rods 41 and the rotating apparatus 3.

When the arm rods 41 connect the motor 32, the movement arm 4 can stretch and rotate, and the stretching direction of the movement arm 4 is consistent with the receiving chamber 303. When an examinee is in the receiving chamber 303, the rotating apparatus 3 may rotate at 360 degrees, and drive the movement arm 4 to rotate round the receiving chamber 303, such that the examinee receives an all-round thorough examination. When an examination region is selected, the rotating apparatus 3 stops rotating, and the positions of the arm rods 41 are adjusted, such that the permanent magnet 5 traverses all positions in the examination region.

As a preferred embodiment of the present invention, the movement arm 4 may comprise a first arm rod 411, a second arm rod 412, and a third arm rod 413 that are connected to each other.

One end of the first arm rod 411 is secured to the rotating apparatus 3; one end of the second arm rod 412 is connected to the other end of the first arm rod 411; and one end of the third arm rod 413 is connected to the other end of the second arm rod 412, and the other end of the third arm rod 413 is connected to the permanent magnet 5. The motor 32 is separately disposed between the first arm rod 411 and the rotating apparatus 3, between the first arm rod 411 and the second arm rod 412, and between the second arm rod 412 and the third arm rod 413.

Specifically, the first arm rod 411 is secured to a side surface of the driven wheel 312 via the installation plate 318, and the motor 32 is disposed between the first arm rod 411 and the driven wheel 312. The motor 32 is separately disposed between the first arm rod 411 and the second arm rod 412, and between the second arm rod 412 and the third arm rod 413. The permanent magnet 5 is disposed on the third arm rod 413. Compared with the movement arm 4 using two arm rods 41, the movement arm 4 using three arm rods 41 can reduce the length of a single arm rod 41, and can more flexibly control the permanent magnet 5 disposed at a tail end.

As another embodiment of the present invention, the movement a 4 has two arm rods: a first arm rod 411, and a second arm rod 412.

One end of the first arm rod 411 is secured to the rotating apparatus 3. One end of the second arm rod 412 is connected to the other end of the first arm rod 411, and the other end of the second arm rod 412 is connected to the permanent magnet 5. The motor 32 is separately disposed between the first arm rod 411 and the rotating apparatus 3, between the first arm rod 411 and the second arm rod 412, and between the second arm rod 412 and the permanent magnet 5. In this way, the third arm rod 413 in the above embodiment is replaced by using the length of the motor 32, the permanent magnet 5 is placed on the output end of the motor 32, and the same effects as the third arm rod 413 are achieved by using the length of the motor 32, thereby saving space.

Referring to FIG. 6, to make capsule endoscope control system more beautiful, and ensure the security of an examinee, the rotating apparatus 3 and movement arm 4 can be covered in a cylinder 304. The cylinder 304 extends along the stretching direction of the movement arm 4, and the receiving chamber 303 penetrates through the cylinder 304 along an axial direction. The movement arm 4 using three arm rods 41 can make the diameter of the cylinder 304 smaller after the length of a single arm rod 41 is reduced. Under the same circumstances of traversing any position of the space of the cylinder 304, the cylinder 304 of the smaller size reduces the volume of entire capsule endoscope control system, which thus reduces space and saves manufacture costs.

Specifically, the motor 32 in the embodiment of the present invention is a servo motor.

The motor disposed between the first arm rod 411 and the driven wheel 312 comprises a first motor 321 and a second motor 322.

The first motor 321 is secured to the driven wheel 312 along a transverse direction, and the output end of the first motor 321 is connected to the second motor 322; and the second motor 322 is disposed along a longitudinal direction, and the output end of the second motor 322 is connected to the first arm rod 411. In this way, the first motor 321 rotates along a transverse axle A at 360 degrees, and the output end of the second motor 322 rotates at 360 degrees along a longitudinal axle B in a vertical plane of the first motor 321.

The motor disposed between the first arm rod 411 and the second arm rod 412 is a third motor 323. The third motor 323 is disposed along a longitudinal direction, and the output end of the third motor 323 is connected to the second arm rod 412. In this way, the output end of the third motor 323 rotates at 360 degrees along a longitudinal axle C in a vertical plane of the first arm rod 411.

The motors disposed between the second arm rod 412 and the third arm rod 413 are a fourth motor 324, a fifth motor 325, and a six motor 326 that are sequentially connected.

The fourth motor 324 is disposed along a longitudinal direction; the fifth motor 325 is disposed along a transverse direction, and connected to the output end of the fourth motor 324; the six motor 326 is disposed along a longitudinal direction, and connected to the output end of the fifth motor 325; and the permanent magnet 5 is disposed on the output end of the six motor 326. In this way, the fourth motor 324 rotates at 360 degrees along a longitudinal axle D in a vertical plane of the second arm rod 412, the fifth motor 325 rotates at 360 degrees along a transverse axle E in a vertical plane of the fourth motor 324, and the six motor 326 drives the permanent magnet 5 along a longitudinal axle F in a vertical plane vertical to the fifth motor 325 to rotate at 360 degrees.

Therefore, by adjusting angles of various motors, and flexibly controlling positions of arm rods 41 and permanent magnet 5, the permanent magnet 5 reaches entire position in the region of the cylinder 304.

A control apparatus 11 runs on the terminal 1, is configured to control running states of the entire capsule endoscope control system, and is communicated with the driving apparatus 2.

The operation interface of the control apparatus 11 comprises various function options. A user may directly operate on the operation interface such as, selecting working mode, or inputting other relevant information, for example, controlling position of the examination couch 8.

In addition, the control apparatus 11 internally contains instruction programs corresponding to various functions on the operation interface. After a user selects a function option, the control apparatus 11 sends the corresponding instruction to the driving apparatus 2. In this way, the user can control running of the capsule endoscope control system by using the control apparatus 11.

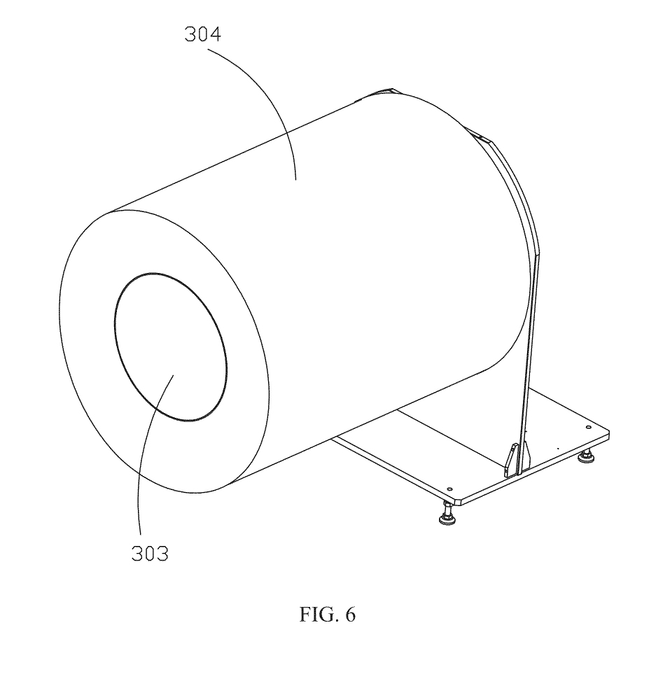

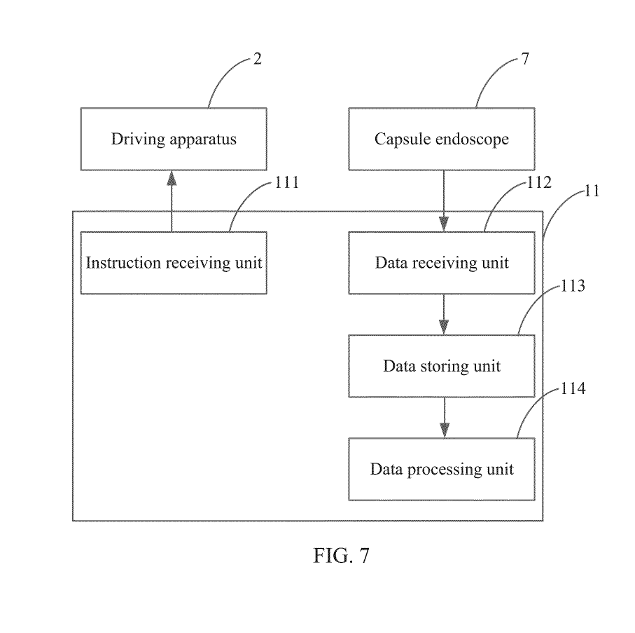

Referring to FIG. 7, the control apparatus 11 comprises: an instruction receiving unit 111, a data receiving unit 112, a data storing unit 113, and a data processing unit 114. The instruction receiving unit 111 is configured to transmit a control instruction input by the user to the driving apparatus 2.

The data receiving unit 112 is configured to receive image data collected and output by a capsule endoscope 7 from a human body.

The data storing unit 113 is configured to store the image data received by the data receiving unit 112.

The data processing unit 114 is configured to process, according to user's operations, the image data such as, browse, edit, and tag, stored by the data storing unit 113.

In an embodiment of the present invention, the terminal 1 is a computer, and may also be another upper-level computer capable of implementing the same functions.

The driving apparatus 2 comprises an input interface, a PLC module, a power module, and an output interface that are electrically connected; wherein: the input interface is configured to receive a movement control instruction input over the terminal 1, the PLC module is configured to perform an operation on the movement control instruction and output a movement control signal, the output interface is configured to output the movement control signal to the rotating apparatus 3 or the movement arm 4, and the power module is configured to supply power to the PLC module.

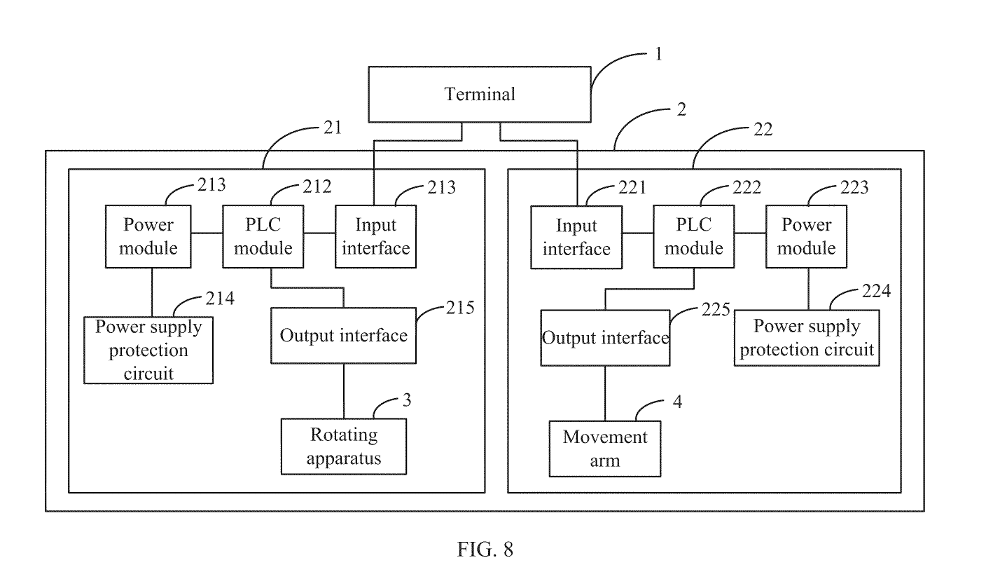

Referring to FIG. 8, the driving apparatus 2 is divided into a rotating apparatus driving module 21, and a movement arm driving module 22. The motor driving the driving wheel 311 is a rotating apparatus motor, and the motor disposed on each arm rod of the movement rod 4 is a movement arm motor.

Specifically, the rotating apparatus driving module 21 internally comprises: an input interface 211, a PLC module 212, a power supply module 213, a power supply protection circuit 214, and an output interface 215.

The input interface 211 is configured to receive instruction information transmitted by the terminal 1, and transmit the instruction information to the PLC module 212. The PLC module 212 internally stores logical operation programs. After receiving an input signal, the PLC module 212 runs the corresponding program according to the input signal, generates an output signal, and finally transmits the output signal to the output interface 215. The output interface 215 transmits the output signal to the rotating apparatus 3 to control rotation of the motor in the rotating apparatus 3.

The power supply module 213 is configured to supply power to the PLC module 212.

The power supply protection circuit 214 is configured to protect the power supply module 213 to prevent exceptions of over-voltage, over-temperature, and over-current.

The movement arm driving module 22 internally comprises: an input interface 221, a PLC module 222, a power supply module 223, a power supply protection circuit 224, and an output interface 225.

The input interface 221 is configured to receive instruction information transmitted by the terminal 1, and transmit the instruction information to the PLC module 222.

The PLC module 222 internally stores logical operation programs. After receiving an input signal, the PLC module 222 runs the corresponding program according to the input signal, and finally transmits an output signal to the output interface 225. The output interface 225 transmits the output signal to the movement arm 4 to control the movement arm 4 to implement corresponding movement.

The power supply module 223 is configured to supply power to the PLC module 222.

The power supply protection circuit 224 is configured to protect the power supply module 223 to prevent exceptions of over-voltage, over-temperature, and over-current.

The output signal transmitted by the rotating apparatus driving module 21 to the rotating apparatus 3 comprises: an angular velocity and rotation angle information of the rotation of the rotating apparatus motor. For example, when a pulse signal is selected as the output signal, the pulse frequency of the pulse signal and the number of pulse signals may be used to correspond to the angular velocity and the rotation angle according to an agreement. After receiving a signal, the rotating apparatus motor rotates at a corresponding angle at a corresponding angular velocity. The rotating apparatus 3 may be subjected to 360 degrees rotation.

The movement arm driving module 22 may transmit a signal to any motor on the movement arm 4. Under driving of the motor, each arm rod may move up, down, left, and right. Therefore, the movement arm 4 moves to a designated position within a specific range under cooperation of the arm rods thereof.

The output signal transmitted by the movement arm driving module 22 comprises such information as movement direction and distance of the arm rods. For example, when a pulse signal is selected as the output signal, the pulse frequency of the pulse signal and the number of pulse signals may be used to correspond to the movement direction and distance of arm rods according to an agreement. Under the cooperative action of the arm rods of the movement rod 4, the movement rod 4 may change the position of the permanent magnet 5 through a series of operations such as shrink and move, and may also control the permanent magnet 5 through a rotation operation to rotate at 360 degrees with a peripheral point as a center of circle, or to self-rotate at 360 degrees with the center of the permanent magnet 5 as an axle. Through the above operations, the movement rod 4 changes the position and posture of the permanent magnet 5.

Referring to FIG. 1, as a preferable embodiment of the present invention, to facilitate examination on an examinee, an examination couch 8 is disposed below the rotating apparatus 3 and the movement arm 4. The examination couch 8 is configured to support an examinee, and may be movably pushed.

The examination couch 8 horizontally passes through the rotating apparatus 3 and is thus placed, and is configured to accommodate the examinee taking a controllable capsule endoscope in the body. After taking a capsule endoscope, the examinee lies down on the examination couch 8. Under the control of the capsule endoscope control system, the permanent magnet 5 may reach a series of different positions around the examinee and stay in different postures. Because the magnetic material in the capsule endoscope is subjected to a magnetic force, the position of the permanent magnet 5 may change under traction of the magnetic force, the movement direction of the capsule endoscope may also change, and finally the capsule endoscope may move to the position corresponding to the permanent magnet 5. If the posture of the permanent magnet 5 changes, the posture of the capsule endoscope may correspondingly change.

The method for controlling capsule endoscope in the body by using the permanent magnet 5 in the capsule endoscope control system is specifically described hereinafter.

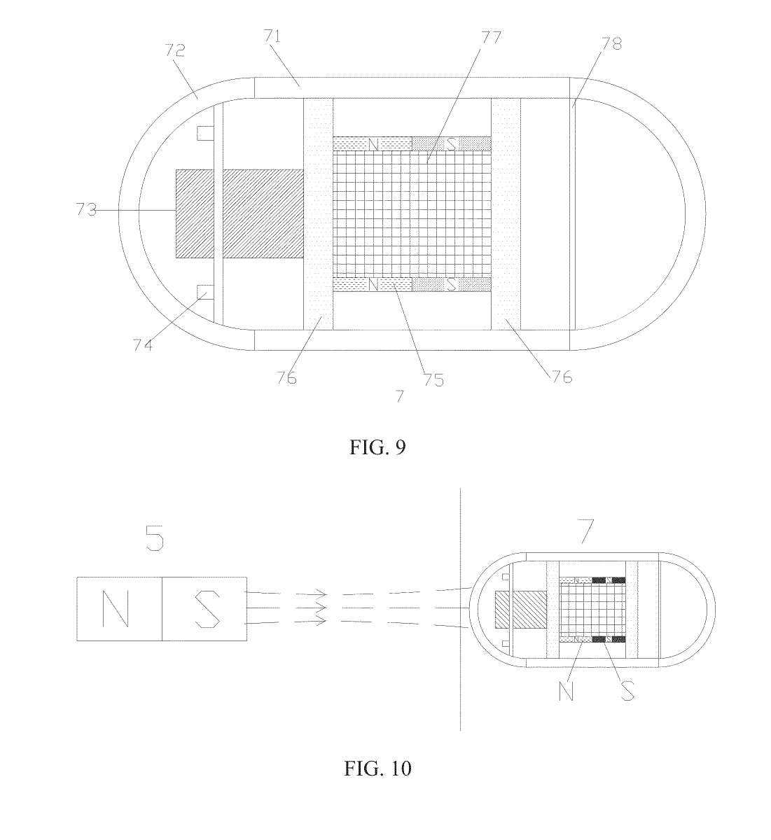

As illustrated in FIG. 9, the capsule endoscope 7 in the embodiment of the present invention comprises: a capsule housing 71, a lens hood 72, and a lens 73 internally disposed therein, an LED light source component 74, a magnet 75, and a circuit board 76, a battery 77, and an antenna 78.

Only a group of lens 73 is selected in the embodiment of the present invention, the lens hood 72 is disposed on a periphery of the lens 73, the battery 77 is connected to the circuit board 76, and the antenna 78 is connected to the circuit board 76. The selected magnetic material in the embodiment of the present invention is magnet 75 disposed on a periphery of the battery 77. The magnet 75 is subjected to a magnetic force in the magnetic field to change the movement state of the capsule endoscope 7.

As illustrated in FIG. 10, the side of the capsule endoscope 7 with a lens needs to be disposed close to the stomach wall when images of the stomach wall in a specific position need to be clearly picked up.

The capsule endoscope control system controls the permanent magnet 5 to be located at a specific position close to the stomach in the human body, and the S pole of the permanent magnet 5 points to the human body, and the N pole points to the opposite. Under traction of the magnetic force, the capsule endoscope 7 moves towards the direction of the S pole of the permanent magnet 5. When moving to the stomach wall close to the S pole of the permanent magnet, the capsule endoscope 7 stops moving. Because the magnetic pole of the magnet has the features of “like charges repel each other while unlike charges attract”, the N pole of the permanent magnet 5 points to the stomach wall, and the N pole points to the opposite. Accordingly, the side of the capsule endoscope 7 with a lens is disposed close to the stomach wall, and the side without a lens is disposed close to the opposite. At this moment, images in a series of the corresponding positions of the stomach wall can be picked up in a short distance. With the same method, the capsule endoscope control system controls the permanent magnet 5 to be located at different positions close to the stomach in the human body, and thus images in a series of the corresponding positions of the stomach wall can be precisely picked up.

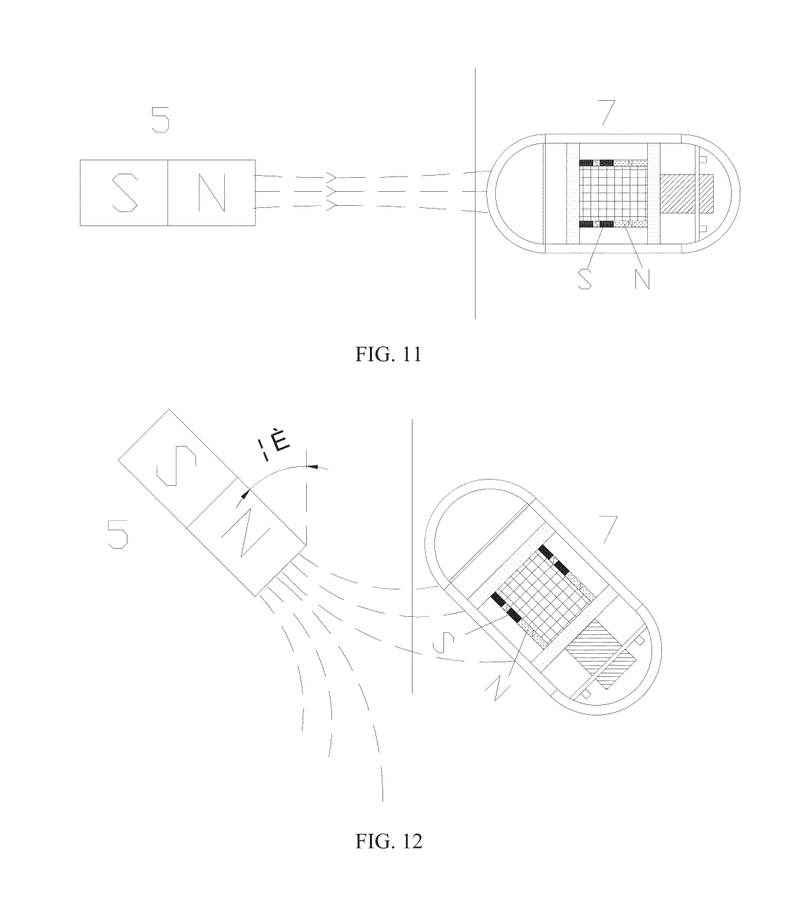

As illustrated in FIG. 11, the side of the capsule endoscope 7 without a lens needs to be disposed close to the stomach wall when a large range of images need to be picked up.

The specific implementing method is as follows: the capsule endoscope control system controls the permanent magnet 5 to be located at a specific position close the stomach in the human body, the N pole points to the human body, and the S pole points to the opposite. Under traction of the magnetic force, the capsule endoscope 7 moves towards the direction of the N pole of the permanent magnet. When moving to the stomach wall close to the N pole of the permanent magnet, the capsule endoscope 7 stops moving, the S pole of the permanent magnet 5 points to the stomach wall, and the N pole points to the opposite. Correspondingly, the side of the capsule endoscope 7 without a lens is disposed close to the stomach wall, and the side with a lens is disposed close to the opposite. At this moment, images within a large range of the stomach wall of the human body can be picked up in a remote distance. With the same method, the capsule endoscope control system controls the permanent magnet 5 to be located in different positions close to the stomach in the human body, and thus images in a series of the corresponding positions of the stomach wall can be picked up.

FIG. 12 illustrates a method for changing postures of the capsule endoscope 7 by changing postures of the permanent magnet 5.

The capsule endoscope control system controls the permanent magnet 5 to be located in a specific position that forms an angle θ(0<θ<180) with the human body. The N pole points to the human body at an angle θ, and the S pole points to the opposite. Under traction of the magnetic force, the capsule endoscope 7 finally moves to the position as illustrated in FIG. 12, the side of the capsule endoscope 7 without a lens points to the stomach wall at the angle θ, and the side with a lens points to the opposite. In this way, the permanent magnet 5 is controlled in different postures by changing the angle θ, which can make the capsule endoscope 7 in different postures, such that the image can be picked up at any angle in the original position.

According to the embodiments of the present invention, by means of electrical control, the driving apparatus controls movement of the rotating apparatus and the movement arm, the permanent magnet disposed on the movement arm controls position and posture of the capsule endoscope in the human body, such that the capsule endoscope picks up complete images on the stomach, and accuracy and precision of medical diagnosis are improved.

Described above are merely preferred embodiments of the present invention, but are not intended to limit the present invention. Any modification, equivalent replacement, or improvement made without departing from the spirit and principle of the present invention should fall within the protection scope of the present invention.

What is claimed is:

1. A capsule endoscope control device, comprising:
a frame;
a rotating apparatus, disposed on the frame;
a movement arm, secured to the rotating apparatus, a tail end of the movement arm being provided with a permanent magnet; and
a driving apparatus, electrically connected to the rotating apparatus and the movement arm, and configured to receive an external movement control instruction, drive the rotating apparatus, the movement arm and the permanent magnet to move, and control position and posture of the capsule endoscope in a human body via a magnetic force of the permanent magnet.

2. The capsule endoscope control device according to claim 1, wherein the frame comprises a base supported on the ground, and a back plate perpendicularly secured to the base along a longitudinal direction; and
the rotating apparatus is secured to the back plate.

3. The capsule endoscope control device according to claim 2, wherein the rotating apparatus comprises:
a driving wheel, securely disposed on one side of the back plate;
a driving wheel motor, securely disposed on the other side of the back plate, and electrically connected to the driving wheel;
a bearing, secured to the back plate; and
a driven wheel, engaged with the driving wheel, and rotatably connected to an outer ring of the bearing.

4. The capsule endoscope control device according to claim 2, wherein the back plate is provided with a through hole;
the bearing is in a hollow annular shape;
wherein the through hole is communicated with a hollow position of the bearing to define a receiving chamber for accommodating an examinee; and
the movement arm is positioned on an outer side of the receiving chamber.

5. The capsule endoscope control device according to claim 3, wherein a guiding plate is secured in an overlay manner to one side of the back plate;
wherein the guiding plate is positioned on an outer ring at a bottom side of the driven wheel; and
an outer edge of the guiding plate extends towards one side of the movement arm to define a circular baffle, a protrusion portion being formed upwardly in a position close to the driven wheel on an inner ring of the guiding plate;
the baffle, the guiding plate, and the protrusion portion defining an annular guiding groove;
the guiding groove being internally provided with a connection cable of the movement arm.

6. The capsule endoscope control device according to claim 3, wherein an installation plate is secured to a top surface of the driven wheel, and one end of the movement arm is secured to the installation plate.

7. The capsule endoscope control device according to claim 1, wherein the movement arm comprises:
a first arm rod, one end of the first arm rod being secured to the rotating apparatus; and
a second arm rod, one end of the second arm rod being connected to the other end of the first arm rod, and the other end of the second arm rod being connected to the permanent magnet;
wherein a motor is separately disposed between the first arm rod and the rotating apparatus, between the first arm rod and the second arm rod, and between the second arm rod and the permanent magnet.

8. The capsule endoscope control device according to claim I, wherein the movement arm comprises:
a first arm rod, one end of the first arm rod being secured to the rotating apparatus;
a second arm rod, one end of the second arm rod being connected to the other end of the first arm rod; and
a third arm rod, one end of the third arm rod being connected to the other end of the second arm rod, and the other end of the third arm rod being connected to the permanent magnet;
wherein a motor is separately disposed between the first arm rod and the rotating apparatus, between the first arm rod and the second arm rod, and between the second arm rod and the third arm rod.

9. The capsule endoscope control device according to claim 1, wherein the driving apparatus comprises an input interface, a PLC module, a power module, and an output interface that are electrically connected; wherein:
the input interface is configured to receive an externally input movement control instruction;
the PLC module is configured to perform an operation on the received movement control instruction and output a movement control signal;
the output interface is configured to output the movement control signal to the rotating apparatus or the movement arm; and
the power module is configured to supply power to the PLC module.

10. The capsule endoscope control device according to claim 1, wherein the driving apparatus comprises:
a rotating apparatus driving module for driving the rotating apparatus; and
a movement arm driving module for driving the movement arm.

11. A capsule endoscope control system, comprising:
a frame;
a rotating apparatus, disposed on the frame;
a movement arm, secured to the rotating apparatus, a tail end of the movement arm being provided with a permanent magnet;
a driving apparatus, electrically connected to the rotating apparatus and the movement arm, and configured to receive an external movement control instruction, drive the rotating apparatus, the movement arm and the permanent magnet to move, and control position and posture of the capsule endoscope in a human body via a magnetic force of the permanent magnet; and
a control apparatus, communicated with the driving apparatus, and configured to provide an operation interface for a user, transmit a movement control instruction of the user to the driving apparatus, store and process image data collected by the capsule endoscope from the human body.

12. The capsule endoscope control system according to claim 11, wherein the frame comprises a base supported on the ground, and a back plate perpendicularly secured to the base along a longitudinal direction; and
the rotating apparatus is secured to the back plate.

13. The capsule endoscope control system according to claim 12, wherein the rotating apparatus comprises:
a driving wheel, securely disposed on one side of the back plate;
a driving wheel motor, securely disposed on the other side of the back plate, and electrically connected to the driving wheel;
a bearing, secured to the back plate; and
a driven wheel, engaged with the driving Wheel, and rotatably connected to an outer ring of the bearing.

14. The capsule endoscope control system according to claim 12, wherein the back plate is provided with a through hole;
the bearing is in a hollow annular shape;
wherein the through hole is communicated with a hollow position of the bearing to define a receiving chamber for accommodating an examinee and
the movement arm is positioned on an outer side of the receiving chamber.

15. The capsule endoscope control system according to claim 13, wherein a guiding plate is secured in an overlay manner to one side of the back plate;
wherein the guiding plate is positioned on an outer ring at a bottom side of the driven wheel; and
an outer edge of the guiding plate extends towards one side of the movement arm to define a circular baffle, a protrusion portion being formed upwardly in a position close to the driven wheel on an inner ring of the guiding plate;
the baffle, the guiding plate, and the protrusion portion defining an annular guiding groove;
the guiding groove being internally provided with a connection cable of the movement arm. 16, The capsule endoscope control system according to claim 13, wherein an installation plate is secured to a top surface of the driven wheel, and one end of the movement arm is secured to the installation plate.

17. The capsule endoscope control system according to claim 11, wherein the movement arm comprises:
a first arm rod, one end of the first arm rod being secured to the rotating apparatus;
a second arm rod, one end of the second arm rod being connected to the other end of the first arm rod, and the other end of the second arm rod being connected to the permanent magnet;
wherein a motor is separately disposed between the first arm rod and the rotating apparatus, between the first arm rod and the second arm rod, and between the second arm rod and the permanent magnet.

18. The capsule endoscope control system according to claim 111, wherein the movement arm comprises:
a first arm rod, one end of the first arm rod being secured to the rotating apparatus;
a second arm rod, one end of the second arm rod being connected to the other end of the first arm rod; and
a third arm rod, one end of the third arm rod being connected to the other end of the second arm rod, and the other end of the third arm rod being connected to the permanent magnet;
wherein a motor is separate disposed between the first arm rod and the rotating apparatus, between the first arm rod and the second arm rod, and between the second arm rod and the third arm rod.

19. The capsule endoscope control system according to claim 11, wherein the driving apparatus comprises an input interface, a PLC module, a power module, and an output interface that are electrically connected; wherein:
the input interface is configured to receive an externally input movement control instruction;
the PLC module is configured to perform an operation on the received movement control instruction and output a movement control signal;
the output interface is configured to output the movement control signal to the rotating apparatus or the movement arm; and
the power module is configured to supply power to the PLC module. 20, The capsule endoscope control system according to claim 11, wherein the driving apparatus comprises:
a rotating apparatus driving module for driving the rotating apparatus; and
a movement arm driving module for driving the movement arm.

21. The capsule endoscope control system according to claim 11, wherein the control apparatus comprises:
an instruction receiving unit, configured to transmit the movement control instruction input by the user to the driving apparatus;
a data receiving unit, configured to receive the image data collected and output by the capsule endoscope from the human body;
a data storing unit, configured to store the image data received by the data receiving unit; and
a data processing unit, configured to process, according to user's operations, the image data stored by the data storing unit.
